(19) United States
(12) Patent Application Publication
(10) Pub. No.: US 2026/0272495 A1
(43) Pub. Date: Sep. 17, 2026

(54) NONINVASIVE MYECTOMY DEVICE AND METHOD

(71) Applicant: Mayo Foundation for Medical Education and Research, Rochester, MN (US)

(72) Inventors: Nishant Saran, Rochester, MN (US); Joseph A. Dearani, Rochester, MN (US); Hartzell V. Schaff, Rochester, MN (US); Samuel J. Asirvatham, Rochester, MN (US); Stephen T. Kuehn, Woodbury, MN (US); Caroline M. Murray, Saint Paul, MN (US)

(21) Appl. No.: 19/472,793

(22) PCT Filed: Apr. 4, 2024
(86) PCT No.: PCT/US2024/023014

§ 371 Date: Oct. 6, 2025

Related U.S. Application Data
(60) Provisional application No. 63/457,605, filed on Apr. 6, 2023.

(51) Int. Cl. A61B 17/32; A61M 25/00; A61M 25/01

(52) U.S. Cl. CPC A61B 17/32002; A61B 2017/320024; A61B 2217/005; A61M 25/0074; A61M 25/0082; A61M 2025/0095; A61M 25/0127

(57) ABSTRACT

The present disclosure relates to a catheter-directed myectomy device. The device includes a catheter comprising a body having a distal end, a first lumen, and a second lumen. A blade assembly is disposed in the second lumen of the catheter and movable relative to the distal end between a retracted position and an extended position. A suction assembly is configured to direct a suction force through the first lumen.

### CROSS-REFERENCE TO RELATED APPLICATIONS

This application claims the benefit of U.S. Provisional Application Ser. No. 63/457,605, filed Apr. 6, 2023. The disclosure of the prior application is considered part of the disclosure of this application, and is incorporated in its entirety into this application.

### TECHNICAL FIELD

The present disclosure relates to a catheter, and more particularly, to a catheter device for performing a septal myectomy.

### BACKGROUND

Hypertrophic cardiomyopathy (HCM) is a disease in which the heart muscle becomes thickened (hypertrophied). The thickened heart muscle can make it harder for the heart to pump blood. A septal myectomy is an open-heart surgical procedure for treating HCM, and involves removing part of the thickened, overgrown septum between the right and left ventricles.

For a septal myectomy, a surgeon typically makes a long cut down the center of the subject's chest along the breastbone, and separates part of the breastbone and/or spreads open the rib cage to access the heart. The surgical team attaches the subject's heart to a heart-lung machine that provides oxygen to the subject's blood and pumps the blood to the subject's body during surgery. With a blade, the surgeon cuts away part of the thickened area of the septum near the base of the heart. After excising the thickened heart muscle, the surgical team weans off and disconnects the heart-lung machine from the patient, wires the breastbone back together, and sews or staples the subject's skin back together at the incision site. Currently, there are no minimally invasive approaches to perform a septal myectomy.

### SUMMARY

The present disclosure relates to devices and methods for performing a minimally invasive septal myectomy.

In a first example aspect, a catheter-directed myectomy device may include a catheter body having a distal end, a first lumen, and a second lumen. A blade assembly may be disposed in the second lumen of the catheter and may be movable relative to the distal end between a retracted position and an extended position. A suction assembly may be configured to direct a suction force through the first lumen.

In a second example aspect, a method of removing tissue from a heart of a subject may include inserting a catheter device through an aortic valve of the subject. The catheter device may include a body having a distal end, an outer sheath, a blade assembly, and a suction assembly. The blade assembly and the suction assembly may be disposed within the outer sheath during insertion. The method may further include fixing the distal end of the catheter device to a tissue at a target site of the heart, and exposing the blade assembly to the tissue. The method may include applying a suction force at the distal end, via the suction assembly.

In a third example aspect, a catheter-directed myectomy device may include a catheter body having a distal end defining an opening and a proximal end opposite the distal end. A handle may be coupled to the proximal end of the catheter body. A suction assembly may be configured to direct a suction force at the opening of the catheter body. A blade assembly may include a blade that is configured to extend through the opening.

In accordance with any one of the first, second, and third example aspects, the catheter-directed myectomy device and the method of removing tissue from a heart of a subject may include any one or more of the following forms.

In one form, a third lumen may be disposed in the second lumen of the catheter body.

In some forms, a fixation assembly may be disposed in the third lumen of the catheter body.

In other forms, the fixation assembly may include a helical anchor that is movable between an extended position and a retracted position.

In another form, the third lumen may be configured to deliver a saline wash through the distal end of the catheter body.

In yet another form, third lumen may be configured to deliver a suction force through the distal end of the catheter body.

In one example, the blade assembly may include a shaft and a blade coupled to the shaft.

In some examples, the shaft may define the third lumen.

In other examples, the catheter body may include a sheath surrounding the blade assembly.

In some examples, the sheath may be movable to expose the blade assembly.

In another example, a porous filter may be coupled to the catheter body and spaced away from the distal end.

In some examples, the filter may be configured to engage an arterial wall when the filter is in an expanded configuration.

In yet another example, the distal end of the catheter body may be maneuverable.

In one aspect, a marker coupled to the catheter body that is visible under fluoroscopy.

In some aspects, the marker may be longitudinally aligned with a port formed in the catheter body.

In other aspects, the suction assembly may include a port in fluid communication with the first lumen and coupled to the catheter body.

In another aspect, fixing the distal end may include inserting an anchor into the tissue, the anchor coupled to the blade assembly.

In yet another aspect, fixing the distal end may include applying a suction force to the target site to couple the distal end to the target site.

In some forms, the method may include inserting the catheter device through a femoral vein before inserting the catheter device through the aortic valve.

In other forms, the method may include monitoring the location of the distal end of the catheter device under fluoroscopy.

In some forms, the catheter device may include a marker disposed at the distal end that is visible under fluoroscopy.

In another form, the method may include delivering saline through the distal end of the body.

In some forms, the saline may be configured to travel through an interior lumen of the catheter device.

In one form, the method may include expanding a filter in an aorta of the subject.

In some forms, the filter may be coupled to the outer sheath of the catheter device and configured to engage an interior wall of the aorta.

In one example, a helical anchor may be movable between an extended position, in which the helical anchor extends through a second opening in the distal end of the catheter body, and a retracted position, in which the helical anchor is entirely disposed within the catheter body.

In another example, a catheter cap may define a tip of the catheter body.

In some examples, the tip may define the second opening.

In other examples, the distal end of the catheter body may include a blade housing defining an interior volume and the opening in the distal end.

In another example, the blade may be disposed in the interior volume.

In one aspect, the suction assembly may include a first lumen in fluid communication with the interior volume of the blade housing.

In another aspect, a second lumen may be configured to deliver a saline wash through the opening of the catheter body.

In some aspects, the second lumen may be in fluid communication with the interior volume of the blade housing.

In other aspects, a pull-wire may extend through the catheter body and may be coupled to the distal end.

In one aspect, the pull-wire may be fixed to the catheter cap.

In another aspect, a guidewire may extend through the catheter body and may be positioned in the ventricle or atrium to guide positioning of the distal end toward a target tissue site.

In one form, an embolic protection device may be configured to move between a stored position, in which the embolic protection device is in a collapsed configuration, and a deployed position, in which the embolic protection device is in a radially expanded configuration relative to the catheter body.

In another form, a motor may be disposed in the handle and may be operably coupled to the blade assembly.

In some examples, the methods and devices described herein provide an alternative to open-heart surgery, thereby reducing health risks and recovery time associated with open-heart surgery.

In some examples, the devices described herein provide a built-in safety device to prevent stroke during the procedure.

Other features and advantages of the present disclosure will be apparent from the following detailed description, the figures, and the claims.

### BRIEF DESCRIPTION OF THE DRAWINGS

FIG. 1 is a perspective view of a catheter-directed myectomy device assembled in accordance with the teachings of the present disclosure;

FIG. 2A is a partial cross-sectional view of the myectomy device of FIG. 1, showing a fixation assembly in a retracted position;

FIG. 2B is a partial cross-sectional view of the myectomy device of FIG. 1, showing the fixation assembly in an extended position;

FIG. 3 is a partially transparent front view of the myectomy device of FIG. 1;

FIG. 4 is an anatomical representation of a heart of a human subject and the myectomy device of FIG. 1 attached to a septum in a left ventricle of the heart;

FIG. 5 is a magnified view of the myectomy device disposed adjacent to the septum of FIG. 4;

FIG. 6 is a magnified view of the myectomy device attached to the septum of FIG. 4;

FIG. 7 is a flow diagram of a method of performing a minimally invasive septal myectomy in accordance with the teachings of the present disclosure;

FIG. 8 is another example of a catheter-directed myectomy device disposed in a heart of a human subject and assembled in accordance with the teachings of the present disclosure;

FIG. 9 is a front view of the myectomy device of FIG. 8;

FIG. 10 is a perspective view of another catheter-directed myectomy device assembled in accordance with the teachings of the present disclosure;

FIG. 11 is a magnified view of the device of FIG. 10, showing an embolic protection device in a stored position;

FIG. 12 is a magnified view of the device of FIG. 10, showing the embolic protection device in a deployed position;

FIG. 13 is a partially transparent, perspective view of a distal end of the device of FIG. 10, showing a blade assembly in a storage position;

FIG. 14 is a bottom view of the distal end of the device of FIG. 10, showing the blade assembly in the storage position;

FIG. 15 is a side view of the distal end of the device of FIG. 10, showing the blade assembly in a use position; and

FIG. 16 is a magnified, cross-sectional view of the distal end of the device of FIG. 10.

### DETAILED DESCRIPTION

The present disclosure relates to catheter devices and methods for performing a minimally invasive septal myectomy. In one example, the device includes a rotating, maneuverable micro blade, a suction cup vacuum tip, and a constant saline wash. The device is configured to excise tissue, flush with a saline wash, and retrieve the tissue and wash from the site.

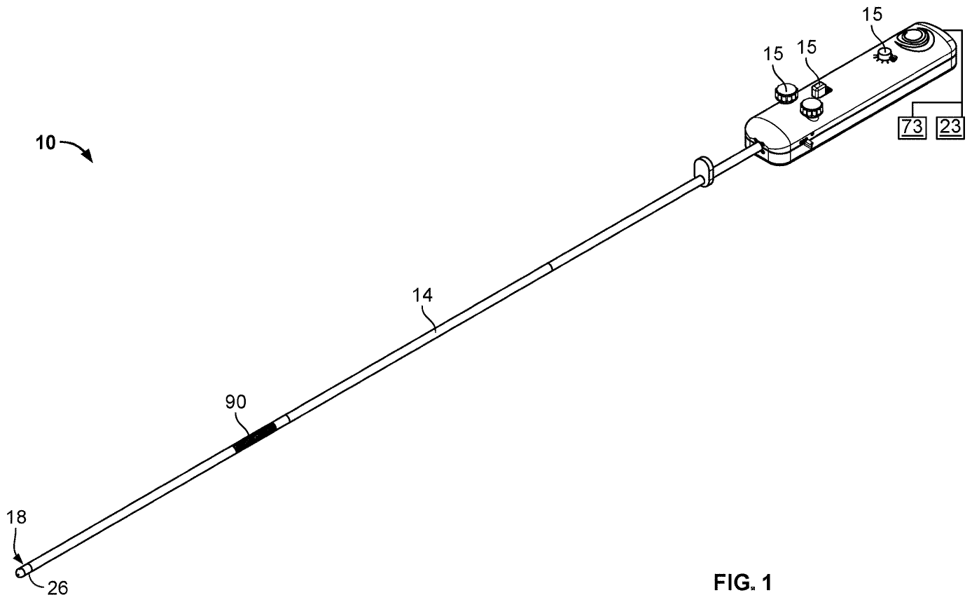

In FIGS. 1-3, an example catheter-directed myectomy device 10 (also referred herein as a “catheter device” or a “myectomy device”) includes a handle 12 at a proximal end, a catheter body 14, and a blade assembly 18 at a distal end. The device 10 includes the blade assembly 18 coupled to the body 14, a suction assembly 22 configured to direct a suction force at a distal end 26 of the body 14, and a fixation assembly 30 configured to attach the distal end 26 to a target tissue site. The distal end 26 of the catheter 14 includes a first opening 34 at a tip of the catheter 14 and a second opening 38 that is perpendicularly disposed relative to the first opening 34. The distal end 26 of the catheter 14 is maneuverable in at least two directions relative to an X axis.

The handle 12 includes a plurality of actuating elements (e.g., buttons, knobs, dials, or slides) 15 configured for manipulating the various features (e.g., suction force, saline delivery, blade operation, catheter delivery configuration, in-use configuration) of the device 10. For example, one or more of the actuating elements 15 are configured for maneuvering the distal end 26 of the catheter 14, actuating the blade assembly 18, operating suction, and/or controlling saline delivery. The handle 12 may also house a battery, a motor, or other actuating components for operating the device 10. As schematically depicted in FIG. 1, vacuum and saline sources 23, 73 are connected to lumens of the device 10 extending out of a proximal end of the handle 12. However, in other examples, the lumens may extend out of a side wall of the handle 12.

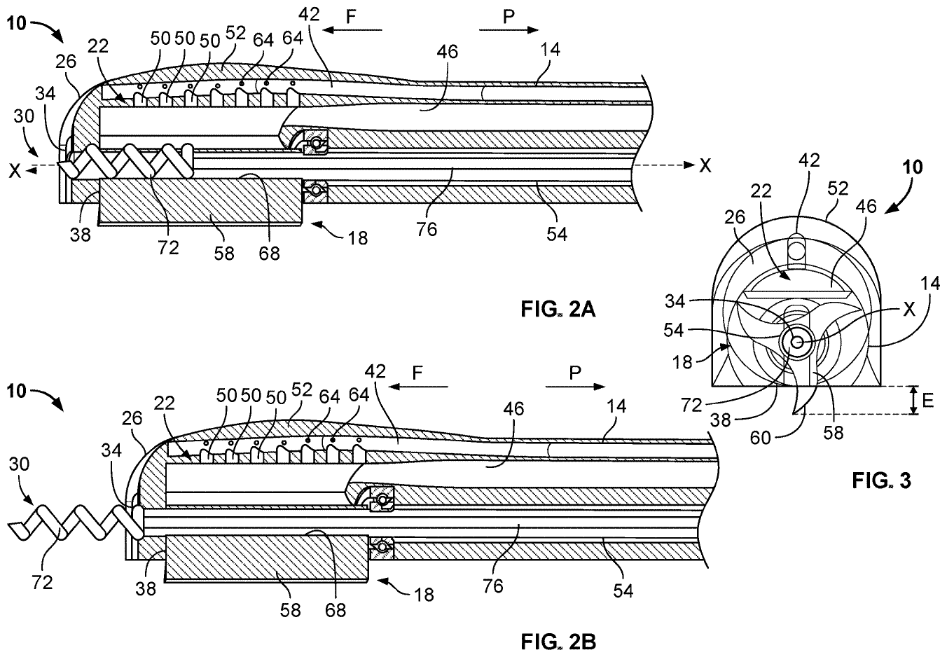

The catheter body 14 includes a first lumen 42 and a second lumen 46 disposed adjacent to the first lumen 42. The blade assembly 18 is disposed in the second lumen 46 and is movable relative to the distal end 26 between a retracted position and an extended position. The suction assembly 22 includes a plurality of ports 50 formed in an outer sheath 52 of the catheter body 14 and is configured to direct a suction force through the first lumen 42. The outer sheath 52 defines the first lumen 42 and at least partially the second lumen 46. As shown in FIGS. 2A and 2B, the outer sheath 52 creates a bulbous distal end with a slightly greater width than a width of the catheter body 14. The catheter size may be in a range of 14 French to 24 French.

The blade assembly 18 includes a shaft 54 and a blade 58 rotatably coupled to the shaft 54. During a procedure, a physician may manually manipulate the blade assembly 18 (e.g., rotating the shaft by hand), or a motor device may actuate the blade assembly 18. The blade 58 includes two or more elements that rotate relative to the X axis. Additionally, the blade assembly 18 is axially movable along the X axis, relative to the catheter body 14, between the retracted and extended positions. To move the blade assembly 18, a physician can either move the outer sheath 52 in a direction P to expose the blade assembly 18 while the blade assembly 18 remains in place, or move the blade assembly 18 in a direction F while the outer sheath 52 remains in place. In FIGS. 2A, 2B, and 3, when the blade assembly 18 is in an extended position, a tip 60 of the blade 58 extends through the second opening 38 of the catheter distal end 26 a distance E. The distance E may have a dimension in a range of approximately 0.8 mm or more (e.g., about 0.81 mm or more, about 0.82 mm or more, about 0.83 mm or more, about 0.84 mm or more, about 0.85 mm or more, about 0.86 mm or more, about 0.87 mm or more, about 0.88 mm or more, about 0.89 mm or more, about 0.9 mm or more, about 0.91 mm or more, about 0.92 mm or more, about 0.93 mm or more, about 0.94 mm or more, about 0.95 mm or more, about 0.96 mm or more, about 0.97 mm or more, about 0.98 mm or more, about 0.99 mm or more, about 1 mm) to approximately 1.2 mm or less (e.g., about 1.19 mm or less, about 1.18 mm or less, about 1.17 mm or less, about 1.16 mm or less, about 1.15 mm or less, about 1.14 mm or less, about 1.13 mm or less, about 1.12 mm or less, about 1.11 mm or less, about 1.1 mm or less, about 1.09 mm or less, about 1.08 mm or less, about 1.07 mm or less, about 1.06 mm or less, about 1.05 mm or less, about 1.04 mm or less, about 1.03 mm or less, about 1.02 mm or less, about 1.01 mm or less, about 1 mm). In the retracted position, the tips 60 of the blade 58 do not extend through the opening 38.

The suction assembly 22 includes a vacuum source 23 (FIG. 1) that is fluidly connected to the first lumen 42 of the catheter body 14. The plurality of ports 50 fluidly connect the first lumen 42 and the second lumen 46 to provide a force of suction at the openings 34, 38 of the distal end 26 of the catheter 14. A plurality of markers 64 that are visible under fluoroscopy are aligned with the ports 50. As the outer sheath 52 is retracted to expose the blade 58, the markers 64 signify a depth of muscle being penetrated by the blade assembly 18. So configured, as the blade assembly 18 pulverizes the targeted tissue, the suction assembly 22 retrieves and removes the pulverized tissue from the target site. The markers 64 identify the position of the catheter 14 under fluoroscopy and potentially may be used with a transesophageal echocardiogram (“TEE”), as well. The markers 64 may also help guide how far the catheter body 14 needs to go along a length of the septum.

The fixation assembly 30 is coupled to the blade assembly 18 and is movable between a retracted position, as shown in FIG. 2A, and an extended position, as shown in FIG. 2B. The fixation assembly 30 is disposed in a third lumen 68 that is defined by the shaft 54 of the blade assembly 18, and includes a helical anchor 72 and a rotatable bit 76. The helical anchor 72 moves independently from the blade assembly 18. When the fixation assembly 30 is actuated, the rotatable bit 76 rotates, causing the helical anchor 72 to rotate as well. So configured, when the fixation assembly 30 extends beyond the tip of the catheter 14, either through the first opening 34 or the second opening 38, the bit 76 rotates to drill the anchor 72 into the target tissue site. At the same time, a physician can move the fixation assembly 30 into the extended position. Once the fixation assembly 30 is sufficiently anchored to the target tissue, the blade assembly 18 may be extended over the fixation assembly 30 to begin excising the target tissue. In some examples, the third lumen 68 is configured to deliver saline from a fluid source 73 and/or a suction force from the vacuum source 23 through the distal end 26 of the catheter 14 during the procedure.

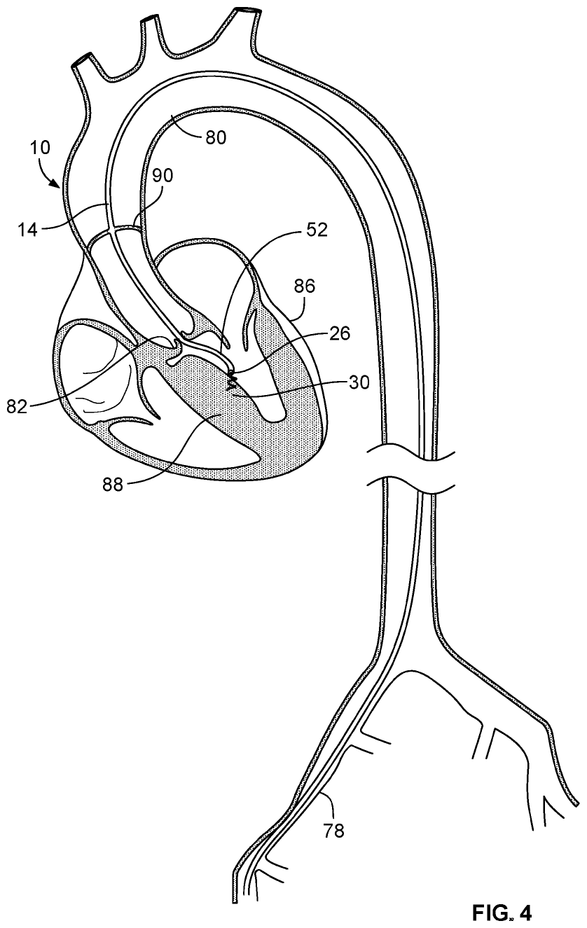

Referring to FIG. 4, the myectomy device 10 is disposed within a body of a subject for performing a septal myectomy. The catheter body 14 extends through a femoral vein 78, into an aorta 80, and through an aortic valve 82 of the subject. The distal end 26 of the catheter 14 is disposed in a left ventricle of a heart 86 of the subject, and is coupled to a tissue site 88 within the heart 86. The fixation assembly 30 is fixed to a hypertrophied tissue segment of the tissue site 88, and the distal end 26 of the catheter 14 is disposed around the assembly 30, providing a vacuum to suction pulverized tissue as the blade assembly 18 advances into the tissue site 88. While the illustrated device 10 is shown through the femoral vein and the aortic valve, the device 10 may be inserted into the patient by other methods including, for example, a peripheral vein or an apex of the heart to reach the target site.

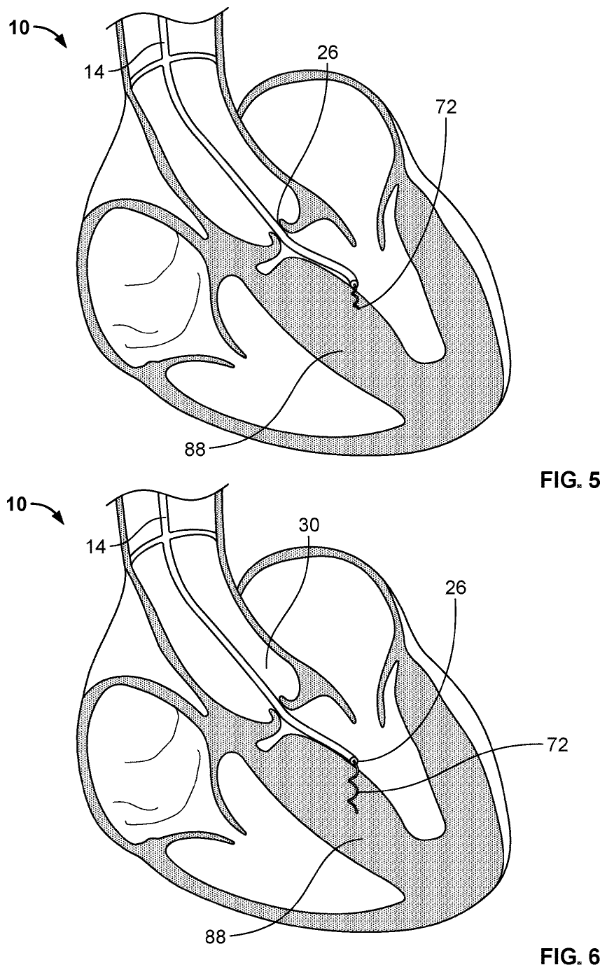

In FIG. 5, before the blade assembly 18 advances and is activated, the distal end 26 of the catheter 14 is placed adjacent to the target tissue site 88 with echocardiographic and fluoroscopic guidance. The fixation assembly 30 extends from the distal end 26 and rotatably and axially advances to anchor into the tissue site 88. Once anchored, the catheter distal end 26 advances over the fixation assembly 30, as shown in FIG. 6, and an embolic protection device 90 expands in the aorta 80. In FIG. 6, the blade assembly 18 is positioned against the tissue target site.

In the aorta 80 shown in FIGS. 4-6, the embolic protection device 90 is coupled to the catheter body 14 and engages with a wall of the aorta 80. The device 90 is a sieve-like filter 90, and is spaced away from the distal end 26 and configured to trap any pulverized muscle from entering the aorta 80. The filter 90 is movable between a collapsed configuration, when the catheter 14 is delivered to the target site, and an expanded configuration, as shown in FIG. 4, when the blade assembly 18 is activated. By catching pulverized muscle in the ascending aorta 80, the filter 90 acts as a stroke-prevention device while permitting blood to flow through the filter pores.

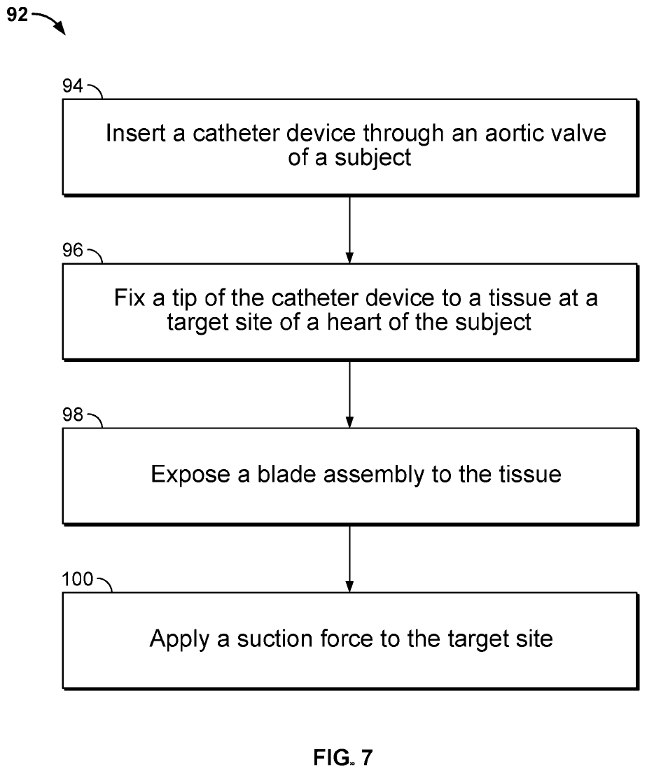

A method 92 of performing a minimally invasive septal myectomy using, for example, the myectomy device 10 of FIGS. 1-6, is shown in FIG. 7. For ease of reference, the method 92 will be described with reference to the subject-inserted myectomy device of FIGS. 1-6. However, the method 92 may be performed using different example myectomy devices. For a human patient, the method 92 may include inserting the device 10 through a femoral vein 78 in the patient's groin, and navigating the distal end 26 of the device 10 to the patient's heart 86. The method 92 includes a step 94 of inserting the catheter device 10 through an aortic valve 82 of the subject, as shown in FIG. 4. The catheter device 10 includes the body 14 having the distal end 26, the outer sheath 52, the blade assembly 18, and the suction assembly 22. The blade assembly 18 and the suction assembly 22 are disposed within the outer sheath 52 during insertion. The device 10 enters a left ventricle of the subject's heart 86 by crossing the aortic valve 82. The method 92 may include using echocardiographic and fluoroscopic guidance to direct the catheter distal end 26 to the desired hypertrophied segment 88 that needs to be excised.

The method 92 includes a step 96 of fixing the distal end 26 of the catheter device 10 to a tissue 88 at a target site of the heart 86, as shown in FIGS. 4 and 5. In this example, the helical anchor 72 of the fixation assembly 30 advances beyond the tip of the catheter body 14 and into the hypertrophied tissue 88. Subsequently, a step 98 of the method 92 includes exposing the blade assembly 18 to the tissue 88 by moving the blade assembly 18 relative to the outer sheath 52 of the body 14. The method 92 includes a step 100 of applying a suction force to the target site of the heart 86, and specifically, around the blade assembly 18 to receive and remove the pulverized tissue from the heart 86.

The method 92 may include a step of monitoring the location of the distal end 26 of the catheter device 10 under fluoroscopy. For example, a plurality of markers 64 disposed in the distal end 26 of the catheter device 10 can help a physician locate the distal end 26 inside the heart, as well as how far the blade assembly 18 advances into the target tissue 88.

The method 92 may also include a step of delivering saline through the distal end 26 of the catheter 14 from the saline source 73. The saline is configured to travel through the interior lumen 68 of the catheter device 10. In this example, the interior lumen 68 is defined by the shaft 54 of the blade assembly 18. The saline may be delivered constantly throughout the procedure to aspirate and retrieve the pulverized muscle. Additionally, the interior lumen 68 may be fluidly coupled to a vacuum source to provide a suction force at the distal end 26 of the device 10.

The method 92 may include expanding the embolic protection device 90 disposed in the aorta 80 of the subject when the distal end 26 of the catheter 14 is disposed in the left ventricle. The filter 90 is coupled to the outer sheath 52 of the catheter device 10 and is configured to engage an interior wall of the aorta 80 after deployed.

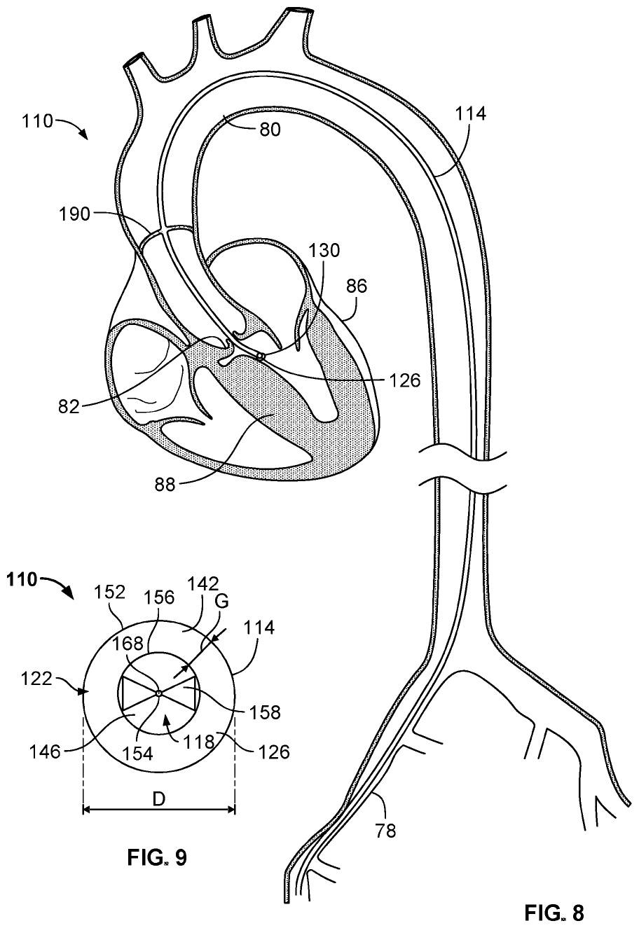

While the blade assembly 18 of the myectomy device 10 of FIGS. 1-16 includes three blades, in other examples, the blade assembly 18 may include two blades, such as a blade assembly 118 of the device of FIGS. 8 and 9.

Referring now to FIGS. 8 and 9, a second example catheter-directed myectomy device 110 may also perform the method 92 of FIG. 7. The second example myectomy device 110 is similar to the myectomy device 10 of FIGS. 1-6, except the second example myectomy device 110 has a different blade assembly 118 and fixation assembly 130. Elements of the second example myectomy device 110 which are similar to the elements of the myectomy device 10 of FIGS. 1-6 are designated by the same reference numeral, incremented by 100. A description of many of these elements is abbreviated or even eliminated in the interest of brevity.

In this example, the catheter distal end 126 includes a distal opening 134 through which the blade assembly 118 extends. As shown in FIG. 9, the catheter 114 includes an exterior sheath 152 defining a first lumen 142, and a first interior sheath 156 defining a second lumen 146. The first and second lumens 142, 146 are axially aligned relative to a Y axis of the catheter device 110, and are radially spaced from each other by a gap G. The gap G may have a dimension in a range of approximately 2 mm or more (e.g., about 2.1 mm or more, about 2.2 mm or more, about 2.3 mm or more, about 2.4 mm or more, about 2.5 mm or more, about 2.6 mm or more, about 2.7 mm or more, about 2.7 mm or more, about 2.8 mm or more, about 2.9 mm or more, about 3.0 mm or more, about 3.1 mm or more, about 3.2 mm or more, about 3.3 mm or more, about 3.4 mm or more, about 3.5 mm) to approximately 5 mm or less (e.g., about 4.9 mm or less, about 4.8 mm or less, about 4.7 mm or less, about 4.6 mm or less, about 4.4 mm or less, about 4.3 mm or less, about 4.2 mm or less, about 4.1 mm or less, about 4.0 mm or less, about 3.9 mm or less, about 3.8 mm or less, about 3.7 mm or less, about 3.6 mm or less, about 3.5 mm).

To both fix the distal end 126 to a target site 88 and also retrieve pulverized tissue during the myectomy procedure, a force of suction is applied at the distal end 126 and through the first lumen 142, between the external and internal sheaths 152, 156 of the catheter body 114. A diameter D of the outer sheath may have a dimension in a range of approximately 1 cm or more (e.g., about 1.01 cm or more, about 1.02 cm or more, about 1.03 cm or more, about 1.04 cm or more, about 1.05 cm or more, about 1.06 cm or more, about 1.07 cm or more, about 1.08 cm or more, about 1.09 cm or more, about 1.1 cm or more, about 1.11 cm or more, about 1.12 cm or more, about 1.13 cm or more, about 1.14 cm or more, about 1.15 cm or more, about 1.16 cm or more, about 1.17 cm or more, about 1.18 cm or more, about 1.19 cm or more, about 1.2 cm or more, about 1.21 cm or more, about 1.22 cm or more, about 1.23 cm or more, about 1.24 cm or more, about 1.25 cm) to approximately 1.5 cm or less (e.g., about 1.49 cm or less, about 1.48 cm or less, about 1.47 cm or less, about 1.46 cm or less, about 1.45 cm or less, about 1.44 cm or less, about 1.43 cm or less, about 1.42 cm or less, about 1.41 cm or less, about 1.4 cm or less, about 1.39 cm or less, about 1.38 cm or less, about 1.37 cm or less, about 1.36 cm or less, about 1.35 cm or less, about 1.34 cm or less, about 1.33 cm or less, about 1.32 cm or less, about 1.31 cm or less, about 1.3 cm or less, about 1.29 cm or less, about 1.28 cm or less, about 1.27 cm or less, about 1.26 cm or less, about 1.25 cm).

The blade assembly 118 is disposed in the second lumen 146, and is exposed by retracting the exterior sheath 152 and/or the interior sheath 156 relative to the blade assembly 118. A blade 158 of the blade assembly 118 includes two opposing elements that rotate relative to the Y axis. Similar to the first example blade assembly 18, the second example blade assembly 118 includes a shaft 154 that defines a third lumen 168. The third lumen 168 can provide a suction force and/or deliver a saline to the target tissue site 88.

Unlike the fixation assembly 30 of the first example myectomy device 10, the fixation assembly 130 of the second example device 110 is provided by the suction assembly 122 and/or an additional suction force. Accordingly, the step 96 of fixing the distal end 126 of the catheter device 110 of method 92 includes applying a suction force through the first lumen 142 to the target site 88 to couple the distal end 126 to the target site 88.

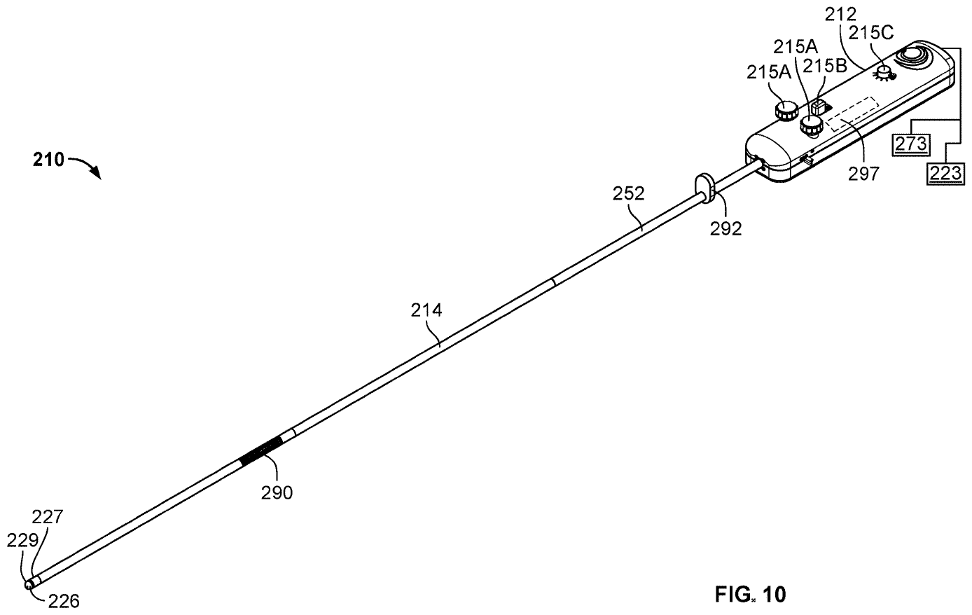

Referring now to FIGS. 10-16, a third example catheter-directed myectomy device 210 may also perform the method 92 of FIG. 6. The third example myectomy device 210 is similar to the myectomy device 10 of FIGS. 1-6. Accordingly, elements of the third example myectomy device 210 which are similar to the elements of the myectomy device 10 of FIGS. 1-6 are designated by the same reference numeral, incremented by 200. A description of many of these elements is abbreviated or even eliminated in the interest of brevity. The third example myectomy device 210 is different from the first and second example myectomy devices 10, 110 as explained below.

In FIGS. 10-13, the myectomy device 210 includes a handle 212, a catheter body 214 coupled to the handle 212, a blade housing 227 disposed at a distal end of the device 10 and coupled to the catheter body 214, and a catheter cap 229 coupled to the blade housing 227. The catheter cap 229 and the blade housing 227 together form a distal end 226 of the myectomy device 210.

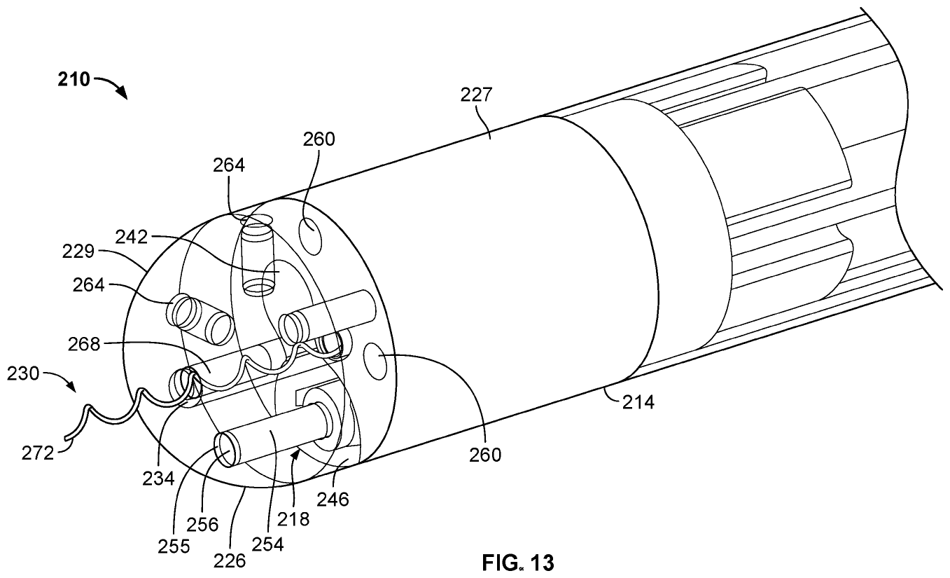

The myectomy device 210 includes a blade assembly 218, a fixation assembly 230, and a suction assembly 222. A user can control each assembly 218, 230, 222 by operating one or more of a plurality of actuating elements 215A, 215B, 215C coupled to the handle 212. The suction assembly 222 includes an external vacuum source 223 and a suction lumen 242 (FIG. 13) that runs through a catheter body 214 and is coupled to the vacuum source 223. The suction assembly 222 is configured to apply a suction force at the distal end 226 of the device 210.

In FIG. 13, the fixation assembly 230 includes a helical anchor 272 and a maneuverable rod (not shown) disposed in an anchor lumen 268 of the catheter body 214. In an extended position, the helical anchor 272 extends through an opening 234 in the distal end 226. The fixation assembly 230 is radially offset relative to the blade assembly 218, and shares the anchor lumen 268 with a guidewire. For example, when delivering the catheter device 210 to the surgical site, a guidewire extends through the anchor lumen 268 and out of the opening 234 of the distal end 226. Once at the surgical site, a user may retract the guidewire from the opening and insert the fixation assembly 230 through the anchor lumen 268.

In other examples, the guide wire may be sufficiently stiff to fix the device 210 next to the target tissue to be excised. Like to the helical anchor 272, a guidewire may be placed in the left ventricle and/or potentially snared from the left atrium and the catheter body 214 could run along the guidewire to excise tissue.

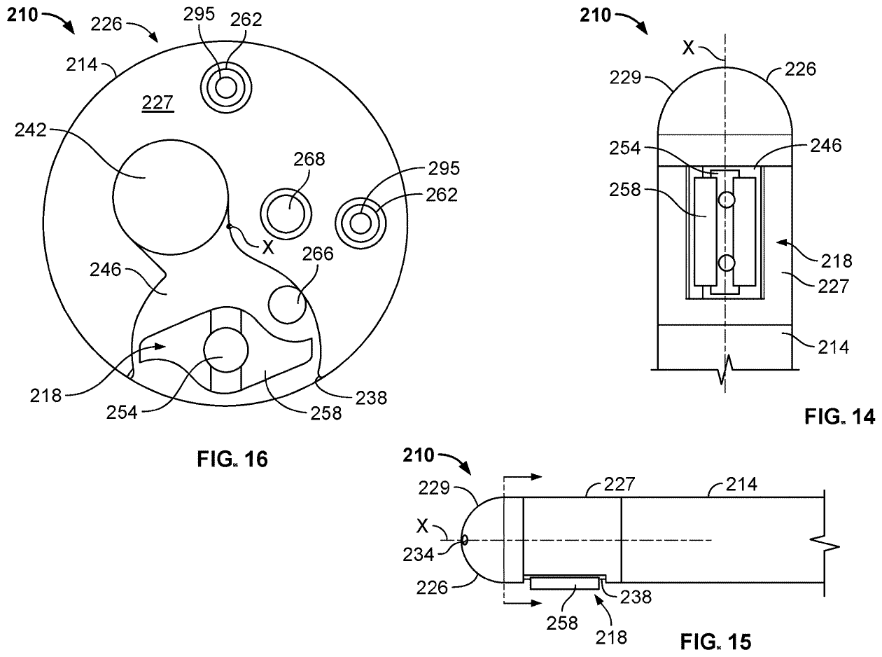

In FIGS. 13-16, the blade assembly 218 includes a rotatable blade shaft 254 and a blade 258 coupled to the blade shaft 254. When the blade assembly 218 is in a storage position, as shown in FIGS. 13 and 14, a distal end 256 of the blade shaft 254 is disposed in a channel 255 formed in the catheter cap 229, and the blade 258 is horizontally oriented relative to an opening 238 in the distal end 226. The blade shaft 254 is coupled to a motor 297 disposed in the handle 212, extends through a blade lumen (not shown) through of the catheter body 214, and terminates in the channel 255. In both the storage position and the in-use position, the blade 258 is disposed in a volume 246 formed in the blade housing 227. The blade 258 is configured to rotate within the volume 246 adjacent to a distal end of the blade lumen. In an in-use position shown in FIG. 15, the blade 258 extends through the opening 238 formed in a sidewall of the catheter body 214.

Returning briefly to FIG. 10, the battery-operated motor 297 for operating the blade assembly 218 is disposed within the handle 212. A drive shaft of the motor 297 is coupled to the blade shaft 254 of the blade assembly 218. The blade assembly 218 may be configured to move from a storage position by pulling a slide 215B in the handle 212, which in turn pulls the blade shaft 254 in the proximal direction away from the channel 255 in the catheter cap 229. The speed of the blade assembly 218 may be controlled by operating one of the actuating elements 215, such as the dial 215C.

The device 210 also includes a wash assembly comprising a flushing lumen 266 and a fluid source 273 (FIG. 10), such as saline, coupled to the flushing lumen 266. As shown in FIG. 16, the flushing lumen 266 and the suction lumen 242 terminate in, and are in fluid communication with, the volume 246 of the blade housing 227. The distal ends of the flushing lumen 266, suction lumen 242, and blade lumen are axially set back relative to the distal ends of the pull wire lumens 262 and anchor lumen 268. The pull wire lumens 262 and the anchor lumen 268 are fluidly isolated from the volume 246 of the blade housing 227. During use, pulverized tissue and saline wash are transferable through the opening 238 at the distal end 226.

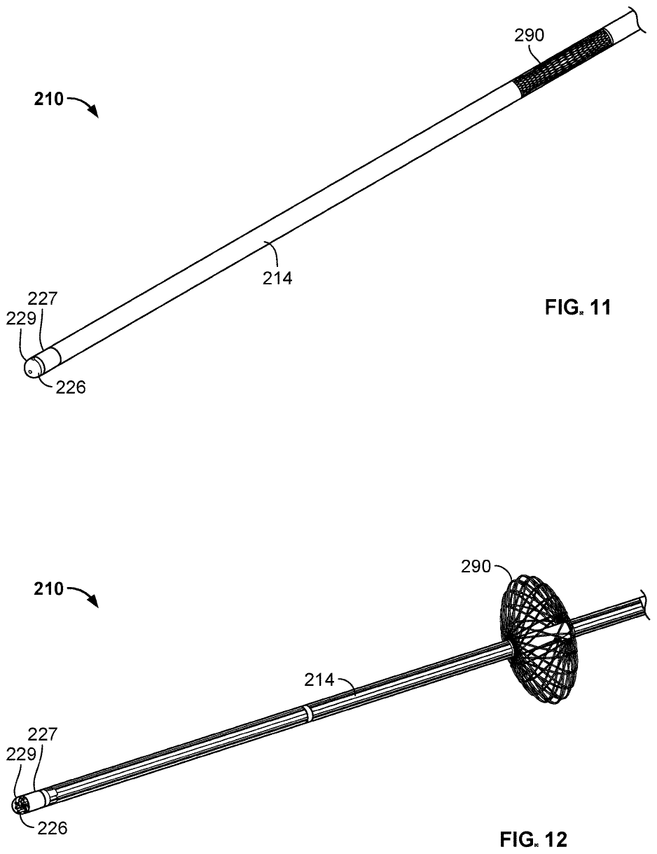

Returning briefly to FIG. 10, the device 210 includes a flange 292 disposed near the handle 212. The flange 292 is coupled to an outer sheath 252 of a catheter body 214, and is configured to move the outer sheath 252 to release an embolic protection device 290. As shown in FIGS. 10 and 11, the embolic protection device 290 is in a collapsed position. When the flange 292 is pushed in a distal direction (i.e., away from the handle 212), a portion of the outer sheath 252 moves in the distal direction and pushes two ends of the embolic protection device 290 to cause the embolic protection device to radially expand, as shown in FIG. 12. To radially collapse the embolic protecting device 290 for storage, a user can pull the flange 292 in a proximal direction (i.e., toward the handle 212), thereby causing the ends of the embolic protection device 290 to move apart.

In FIG. 12, the embolic protection device 290 expands radially outwardly relative to the catheter body 214. When the embolic protection device 290 is deployed inside an artery of a patient, the device 290 radially expands to engage the interior walls of the artery. The embolic protection device 290 includes a mesh, webbed body defining a plurality of openings to permit blood to flow through the device 290, while catching tissue fragments larger than 1 mm.

In FIG. 16, the device 210 includes two pull-wires 295, each disposed in a pull-wire lumen 262 that is radially offset from the central axis X to control navigation of the device. A distal end of each of the pull-wires 295 is fixed to the end 226 of the catheter 214 to maneuver the distal end 226 in at least two directions relative to an X axis. Each pull-wire 295 extends through the catheter body 214 and is coupled to an actuating element 215, such as a knob 215A. By turning the knob 215A in a clockwise direction, for example, tension may be added to the pull-wire 295, and by turning the knob in a counter-clockwise direction, for example, tension may be removed from the pull-wire 295. The pull-wires 295 are used to navigate the device 210 to the target site and to position the blade assembly 218 against the target tissue. To position the blade assembly 218 against the target tissue, the operator can manipulate the pull-wires 295 after the anchor 272 is extended into the tissue. The distal end 226 bends to position the opening 238 of the device 210 against the target site (e.g., FIG. 6). An operator can also rotate the device 210 when or after the anchor 272 extends into the tissue.

While the myectomy device 10 of FIGS. 1-6 includes a fixation assembly coaxially aligned with the blade assembly 18, in other examples, a myectomy device may include a fixation assembly that is offset relative to a blade assembly, such as the example myectomy device 210 as shown in FIGS. 13 and 16.

While the third lumen 68 of the catheter device 10 of FIGS. 1-6 is defined by the blade shaft 54, in other examples, the blade shaft 54 does not define a lumen for the fixation assembly. For example, the fixation assembly 230 of the example catheter device 210 of FIGS. 10-16 is disposed in the anchor lumen 268.

While the myectomy devices 10, 110 of FIGS. 1-6 and FIGS. 8 and 9 each have a blade assembly 18, 118 that is covered by an outer sheath when not in use, in other examples, a myectomy device may include a blade assembly 218 that can be positioned in a storage position without being covered by an outer sheath, such as in the example myectomy device 210 of FIGS. 10-16. As shown in FIGS. 14-16, the blade 258 includes two bladed arms that are positioned 180 degrees relative to each other. In this way, the blade 258 can remain horizontal (relative to an X axis) as shown in FIGS. 14 and 16, and within the catheter 214 when moving the catheter toward the incision site.

While the myectomy device 10 of FIGS. 1-6 includes a plurality of markers 64 aligned with a plurality of ports 50 of the suction assembly 22, in other examples, the device may include one or more markers disposed at other locations in the distal end 226 of the device. For example, the example myectomy device 210 of FIG. 13 includes first and second markers 264 disposed in the catheter cap 229.

While the myectomy device of FIGS. 1-6 delivers saline through the third lumen 68, in other examples, a myectomy device may have a separate flushing lumen, such as the device 210 of FIGS. 10-16. In FIG. 16, the flushing lumen 266 is configured to deliver saline and/or a suction force through the distal end 226 of the catheter 214 during a myectomy procedure. The flushing lumen 266 is in fluid communication with the suction lumen 242, the volume 246 of the blade housing 227, and the opening 238 at the distal end 226 of the device 210.

In another example, a myectomy device may provide a different fixation assembly. For example, the myectomy device may be equipped with an additional suction assembly instead of the helical anchor. In some examples, attachment to the hypertrophied segment 88 or the desired tissue mass can be achieved with one or more devices for performing cryotherapy, such as, for example, a barbed anchor, suction, helical anchor, etc. With cryotherapy, the catheter can attach to the target area on the septum and expose the tissue to freezing temperatures to excise the tissue. In other examples, the fixation device may extend from a lateral side opening of the distal end of the catheter device rather than through an opening in the distal tip.

While each of the myectomy devices 10, 110, 210 includes an expandable filter 90, 190, 290 in other examples, the filter 90, 190, 290 may be optionally attached to the catheter body 14, 114, 214 when it is likely that tissue fragments are less than 1 mm, and do not pose a serious threat of causing a stroke. However, when tissue fragments are larger than 1 mm, the filter 90, 190, 290 may be coupled to the device 10, 110, 210 to expand when in place in the aorta 80 to catch such fragments.

Further treatment of the desired tissue segment 88 may also be achieved by applying direct current use or radiofrequency ablation.

While the devices 10, 110, 210 and method 92 are described herein to perform a septal myectomy procedure, the devices 10, 110, 210 and method 92 may be used in other procedures. For example, the devices 10, 110, 210 may be used in removal of solid organ tumor with localized precision such as from the gastrointestinal tract, liver, kidneys etc.; cardiac tumor removal such as LA myxoma and other tumors; or removal of bronchial tumors and lung parenchymal tumors.

The method 92 and devices 10, 110, 210 described herein may be used via a peripheral approach through peripheral arteries with sufficient caliber such as femoral artery, femoral vein, iliac artery, subclavian artery, carotid artery, direct aortic and/or via transapical (through the apex of the heart) from the left chest. For example, if the target site is approached via a transapical method, the catheter does not need to traverse the aortic valve.

While this specification contains many specific implementation details, these should not be construed as limitations on the scope of any disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A catheter-directed myectomy device, the device comprising:
a catheter body having a distal end, a first lumen, and a second lumen;
a suction assembly configured to direct a suction force through the first lumen; and
a blade assembly disposed in the second lumen of the catheter and movable relative to the distal end between a retracted position and an extended position.

2. The device of claim 1, comprising a third lumen disposed in the second lumen of the catheter body.

3. The device of claim 2, comprising a fixation assembly disposed in the third lumen of the catheter body, wherein the fixation assembly comprises a helical anchor that is movable between an extended position and a retracted position.

4. (canceled)

5. The device of claim 2, wherein the third lumen is configured to deliver a saline wash and a suction force through the distal end of the catheter body.

6. (canceled)

7. The device of claim 2, wherein the blade assembly comprises a shaft and a blade coupled to the shaft, and wherein the shaft defines the third lumen.

8. (canceled)

9. The device of claim 1, wherein the catheter body comprises a sheath surrounding the blade assembly, the sheath being movable to expose the blade assembly.

10. The device of claim 1, comprising a porous filter coupled to the catheter body and spaced away from the distal end, wherein the filter is configured to engage an arterial wall when the filter is in an expanded configuration.

11. (canceled)

12. The device of claim 9, comprising a marker coupled to the catheter body that is visible under fluoroscopy, and wherein the marker is longitudinally aligned with a port formed in the catheter body.

13. (canceled)

14. The device of claim 1, wherein the suction assembly comprises a port in fluid communication with the first lumen and coupled to the catheter body.

15. A method of removing tissue from a heart of a subject, the method comprising:
inserting a catheter device through an aortic valve of the subject, wherein the catheter device comprises a body having a distal end, an outer sheath, a blade assembly, and a suction assembly, wherein the blade assembly and the suction assembly are disposed within the outer sheath during insertion;
fixing the distal end of the catheter device to a tissue at a target site of the heart;
exposing the blade assembly to the tissue; and
applying a suction force at the distal end, via the suction assembly.

16. (canceled)

17. (canceled)

18. (canceled)

19. The method of claim 15, comprising monitoring a location of the distal end of the catheter device under fluoroscopy, wherein the catheter device comprises a marker that is visible under fluoroscopy and that is disposed at the distal end of the catheter device.

20. (canceled)

21. The method of claim 15, comprising deploying an embolic protection device in an aorta of the subject, the embolic protection device coupled to the outer sheath of the catheter device and configured to engage an interior wall of the aorta.

22. The method of claim 15, wherein exposing the blade assembly comprises moving the blade assembly relative to the outer sheath of the body.

23. A catheter-directed myectomy device, the device comprising:
a catheter body having a distal end defining an opening and a proximal end opposite the distal end;
a handle coupled to the proximal end of the catheter body;
a suction assembly configured to direct a suction force at the opening of the catheter body; and
a blade assembly comprising blade that is configured to extend through the opening.

24. The device of claim 23, comprising a helical anchor that is movable between an extended position, in which the helical anchor extends through a second opening in the distal end of the catheter body, and a retracted position, in which the helical anchor is entirely disposed within the catheter body.

25. (canceled)

26. The device of claim 23, wherein the distal end of the catheter body comprises a blade housing defining an interior volume and the opening in the distal end, and wherein the blade is disposed in the interior volume.

27. The device of claim 26, comprising:
a first lumen of the suction assembly in fluid communication with the interior volume of the blade housing; and
a second lumen that is configured to deliver a saline wash through the opening of the catheter body,
wherein the second lumen is in fluid communication with the interior volume of the blade housing.

28. (canceled)

29. (canceled)

30. The device of claim 24, comprising:
a catheter cap defining a tip of the catheter body, the tip defining the second opening; and a pull-wire extending through the catheter body and coupled to the distal end,
wherein the pull-wire is fixed to the catheter cap.

31. (canceled)

32. The device of claim 23, comprising an embolic protection device configured to move between a stored position, in which the embolic protection device is in a collapsed configuration, and a deployed position, in which the embolic protection device is in a radially expanded configuration relative to the catheter body.

33. The device of claim 23, comprising a motor disposed in the handle and operably coupled to the blade assembly.

34. (canceled)
